(19) United States
(12) Patent Application Publication
(10) Pub. No.: US 2021/0220548 A1
(43) Pub. Date: Jul. 22, 2021

(54) SELF-FLUSHING PORTS

(71) Applicant: NP Medical Inc., Clinton, MA (US)

(72) Inventor: Ian Kimball, Townsend, MA (US)

(21) Appl. No.: 17/055,519

(22) PCT Filed: Jun. 12, 2019
(86) PCT No.: PCT/US2019/036743

§ 371 Date: Nov. 13, 2020

Related U.S. Application Data
(60) Provisional application No. 62/684,499, filed on Jun. 13, 2018.

(51) Int. Cl. A61M 5/14; A61M 39/10; A61M 25/02; A61M 39/22

(52) U.S. Cl. CPC A61M 5/14; A61M 39/10; A61M 2005/1403; A61M 39/221; A61M 25/02

(57) ABSTRACT

A fluid conduit has a housing with a body forming an interior and a proximal cavity within the interior. The housing has an outlet and a fluid path extending between the cavity and outlet. The conduit also has an entry channel and a contact surface. The entry channel extends through the housing distal to the cavity and has a radial longitudinal axis. The entry channel is offset from the fluid path such that the radial longitudinal axis of the entry channel does not intersect a longitudinal axis of the fluid path. This offset causes fluid entering the interior of the housing via the entry channel to initiate a swirl-like motion within the interior. A portion of the contact surface is distal to the cavity and intersects the radial longitudinal axis. The surface directs a portion of the fluid proximally into the cavity.

### PRIORITY

This patent application claims priority from U.S. provisional patent application No. 62/684,499, filed Jun. 13, 2018 entitled, “SELF-FLUSHING PORTS,” assigned attorney docket number 130974-04801 (formerly 1600/A48), and naming Ian Kimball as inventor, the disclosure of which is incorporated herein, in its entirety, by reference.

### FIELD OF THE INVENTION

The invention generally relates to medical fluid ports and, more particularly, the invention relates to flushing medical fluid ports.

### BACKGROUND OF THE INVENTION

Many patient fluid transfer applications require a medical practitioner to administer fluid to or take a sample of blood or fluid from the patient through an indwelling catheter. To that end, the practitioner typically uses a fluid transfer set having a sample port that allows the medical practitioner to deliver to or draw a sample of the blood or fluid from the patient's indwelling catheter.

In general terms, medical connectors, such as valving devices, often act as a port that may be repeatedly accessed to non-invasively inject fluid into (or withdraw fluid from) a patient's vasculature. Consequently, a medical connector permits the patient's vasculature to be freely accessed without requiring the patient's skin to be repeatedly pierced by a needle. Alternatively, medical connectors may act as a port for other medical applications, such as for accessing fluid containers (e.g., bags, vials), trachea tubes, enteral lines, breathing apparatuses, surgical sites, etc.

Medical personnel insert a medical instrument into the medical connector to inject fluid into (or withdraw fluid from) a patient who has an appropriately secured medical connector. Once inserted, fluid may be freely injected into or withdrawn from the patient.

One such medical connector/medical fluid delivery system is a “T” type connector that may be utilized within the fluid path. One point of entry to the “T” connector may be a needle free connector, and another point of entry may be an extension set, or short length of tubing, and the remaining port of the “T” would be the exit path or “outlet,” which would lead to the patient (See Figured 1A-1D). Often, the clinician should be able to clear the “T” port of residual fluid (medication, blood, etc.) through a flushing operation. Flushing is not typically a challenge when connecting to a needle free connector and pushing fluid straight through the “T” connector. However, when flushing through the extension set tubing, it is difficult to flush the needle free connector portion of the system, as it is not naturally within the direction of flow. For example, as shown in FIGS. 2A and 2B, fluid entering through the extension set flows into the housing of the connector and then down through the outlet, creating a dead zone within the proximal portion of the connector.

### SUMMARY OF VARIOUS EMBODIMENTS

In accordance with one embodiment of the present invention, a fluid conduit for use in a medical line may have a housing with a body forming an interior. The housing may have a proximal cavity within the interior, an outlet, and a fluid path (with a fluid path longitudinal axis) within the interior and extending between the cavity and outlet. An entry channel may extend through the body of the housing distal to the cavity and may have a radial longitudinal axis. The entry channel may be offset from the fluid path such that the radial longitudinal axis of the entry channel does not intersect the longitudinal axis of the fluid path. This, in turn, may cause at least a portion of a fluid entering the interior of the housing via the entry channel to initiate a swirl-like motion within the interior of the housing. The housing may also have a contact surface within the fluid path. At least a portion of the contact surface may be located distal to the cavity and may intersect the radial longitudinal axis. The contact surface may direct at least a portion of the fluid entering the interior of the housing via the entry channel proximally into the cavity. The entry channel may be configured at an angle such that fluid entering the interior of the housing is directed proximally towards the cavity. In some embodiments, the fluid conduit may include a tubing set having a tube and a medical connector (e.g., a female luer connector). The tube may have a first end and a second end. The tube may be fluidly connected to the entry channel at the first end and the medical connector may be located at the second end. The medical connector may connect to a medical implement (e.g., for introducing fluid into the tubing set). The tubing set may also have a tubing clamp located on the tube. The clamp may transition between an open mode that allows fluid to flow through the tube and a closed mode that prevents fluid from flowing through the tube.

In other embodiments, the contact surface may be angled. For example, the fluid path may include a conical portion with an inner diameter that decreases towards a distal end. The contact surface may be located on the conical portion. Additionally or alternatively, the fluid conduit may include a ramp located within the fluid path and the contact surface may be located on the ramp. The ramp may extend from an inner wall of the housing and into the flow path or the ramp may be recessed into a wall of the housing such that the ramp does not extend into the fluid path. The ramp may be configured at an angle such that fluid entering the interior of the housing is directed proximally towards the cavity. The ramp may extend approximately 180 degrees around the fluid path. In other embodiments, the fluid connector may have a shelf located within the fluid path and distal to the entry channel and the contact surface may be located on a top surface of the shelf.

In accordance with further embodiments, the fluid conduit may have a stabilization pad extending from a portion of the housing. The stabilization pad may stabilize the fluid conduit on a patient during use. Additionally or alternatively, the conduit may have a valve member located within the housing interior. The valve member may have a septum/proximal portion that closes a proximal opening when the valve member is in a closed mode and a valve wall that extends from the septum to form a valve interior. The housing may have an inlet housing and an outlet housing. The valve member may be located at least partially within the inlet housing, and the entry channel may extend through a wall of the outlet housing. In such embodiments, the offset and the contact surface may cause a portion of the fluid to flow into the valve interior.

In accordance with further embodiments, a method of flushing a fluid conduit includes providing a fluid conduit having a housing with a body forming an interior. The housing may also include a cavity within the interior, an outlet, and a fluid path within the interior and extending between the cavity and outlet. The fluid path may have a fluid path longitudinal axis. The fluid conduit may also have an entry channel and a contact surface. The entry channel may extend through the body of the housing and distal to the cavity. The entry channel may also have a radial longitudinal axis and may be offset from the fluid path such that the radial longitudinal axis of the entry channel does not intersect the longitudinal axis of the fluid path. The contact surface may be within the fluid path and at least a portion of the contact surface may be located distal to the cavity and intersect the radial longitudinal axis.

The method may also include connecting a medical implement to a medical connector on a tubing set connected to the entry channel and introducing a fluid into the fluid conduit via the tubing set and the entry channel. The offset may cause at least a portion of the fluid entering the fluid conduit to initiate a swirl-like motion within the fluid path. The contact surface may direct a portion of the fluid proximally into the cavity. In some embodiments, the entry channel may be configured at an angle such that fluid entering the interior of the housing via the entry channel is directed proximally towards the cavity.

In accordance with other embodiments, the contact surface may be angled. For example, the fluid path may include a conical portion and the contact surface may be located on the conical portion. Additionally or alternatively, the fluid conduit may include a ramp located within the fluid path and the contact surface located on the ramp. The ramp may extend from an inner wall of the housing and into the flow path or the ramp may be recessed into a wall of the housing such that the ramp does not extend into the fluid path. The ramp may be configured at an angle such that fluid entering the interior of the housing is directed proximally towards the cavity.

The fluid connector may include a valve member located within the housing interior. The valve member may have a septum configured to close a proximal opening when the valve member is in a closed mode and a valve wall extending from the septum to form a valve interior. The housing may have an inlet housing and an outlet housing. The valve member may be located at least partially within the inlet housing, and the entry channel may extend through a wall of the outlet housing. The offset and the contact surface may cause a portion of the fluid to flow into the valve interior.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIGS. 1A-1D schematically show a prior art T-connector.

FIGS. 2A-2B schematically show cross-sectional views of the prior art T-connector of FIGS. 1A-1D including the flow through the T-connector and the dead zone within the T connector.

FIGS. 3A-3F schematically show various views of a fluid conduit in accordance with various embodiments of the present invention.

FIGS. 4A-4D schematically show various views of an outlet housing of the fluid conduit shown in FIGS. 3A-3F in accordance with some embodiments of the present invention.

FIGS. 5A-5D schematically show various views of an alternative outlet housing of the fluid conduit shown in FIGS. 3A-3F in accordance with additional embodiments of the present invention.

FIGS. 6A-6F schematically show various views of an additional alternative outlet housing of the fluid conduit shown in FIGS. 3A-3F in accordance with further embodiments of the present invention.

FIGS. 7A-7D schematically show fluid flow and flushing within a prior art T-connector and fluid conduits in accordance with embodiments of the present invention.

### DESCRIPTION OF ILLUSTRATIVE EMBODIMENTS

In illustrative embodiments, a self-flushing fluid conduit (e.g., a medical fluid connector or a T-connector) for use in a medical line includes a housing with a proximal cavity within the interior. An entry channel extends through the body of the housing distal to the cavity and is offset from a fluid path extending between the cavity and an outlet. Fluid entering the fluid conduit via the entry channel begins to at least partly swirl within the interior of the housing and contacts a contact surface which, in turn, directs at least a portion of the fluid entering the interior of the housing via the entry channel proximally toward the cavity. This improves flushing of the cavity and the interior of a valve member within the conduit (if equipped).

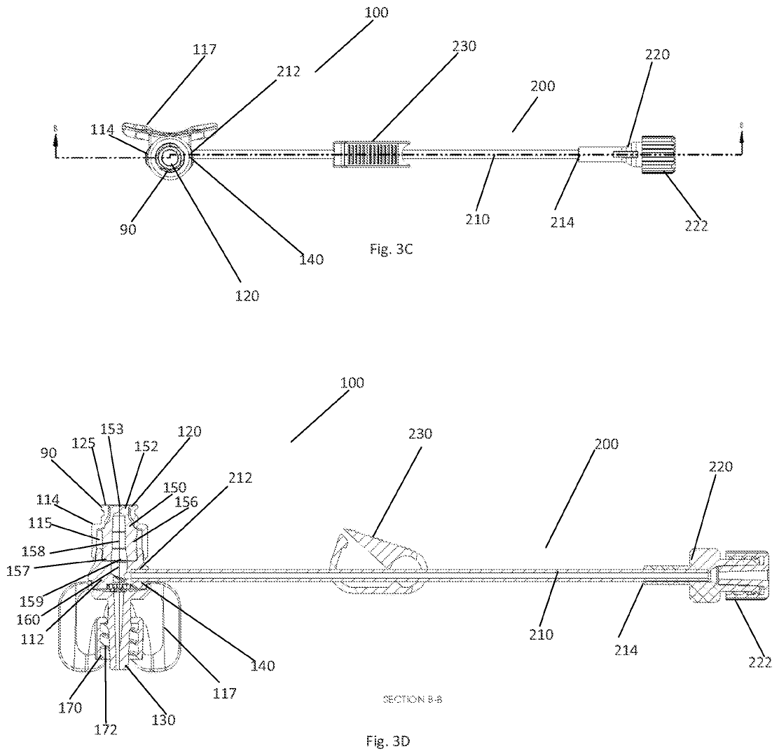

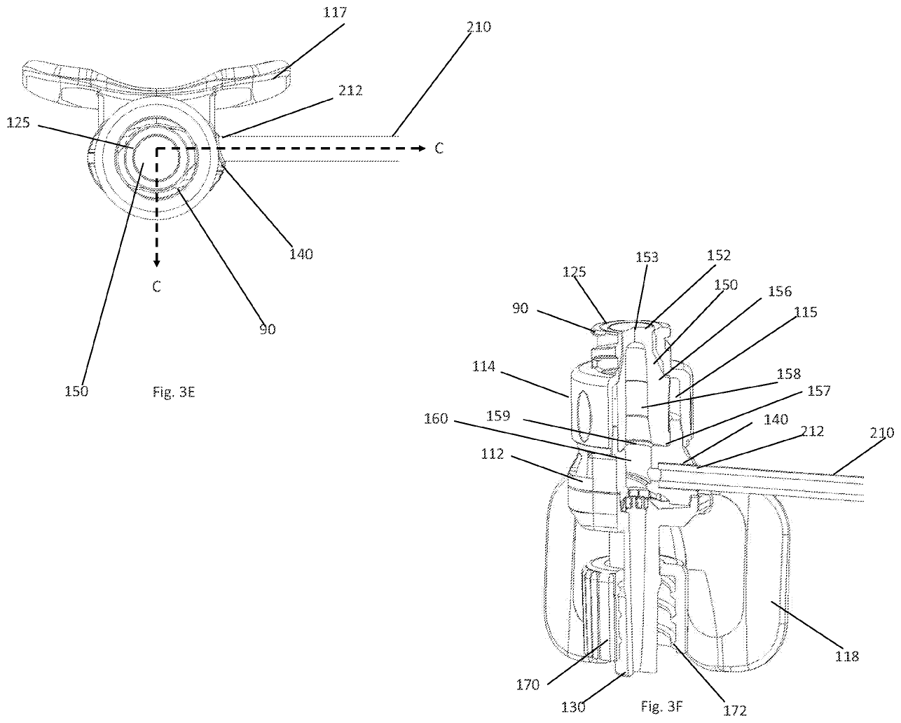

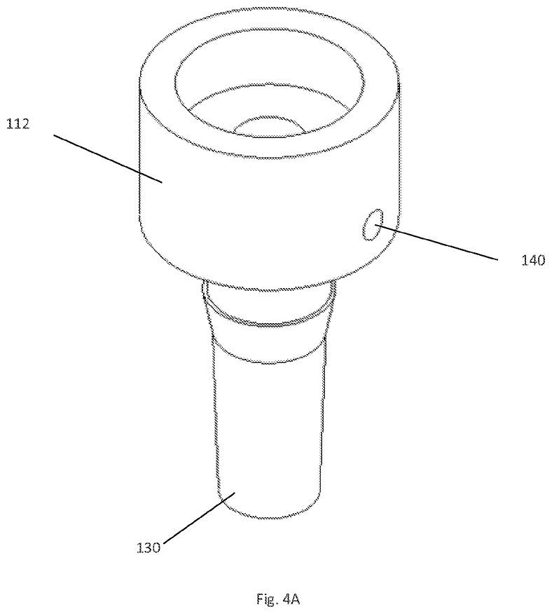

FIGS. 3A-3F schematically show various views of a fluid conduit/medical fluid connector 100 (e.g., a “T” connector) configured in accordance with illustrative embodiments of the invention. The conduit/connector 100 includes a main housing 110 and a tubing set 200 fluidly connected to the main housing 110 at an entry/tubing set fluid channel 140 extending through the main housing 110 (see FIG. 3D). The tubing set 200 includes a tube 210 and a medical connector 220 (e.g., a female luer connector). The tube 210 may be fluidly connected to the entry channel 140 at a first end 212 of the tube 210 and the medical connector 220 may be located at the second end 214 of the tube. In some embodiments, the medical connector 220 may include a cap 222 to prevent contamination within the tube 210 prior to use.

To selectively prevent and allow fluid flow through the tube 210 during use, the tubing set 200 may include a clamp 230 on the tube 210. The clamp 230 may have an open mode that allows fluid flow through the tube 210 and a closed mode that prevents fluid flow through the tube 210, for example, by pinching the tube 210 closed. To that end and as discussed in greater detail below, when the user wishes to flow fluid through the tubing, they may open the clamp 230 (e.g., if it is not already open). Conversely, if the user wishes to prevent fluid flow, the user may close the clamp 230.

As shown in FIGS. 3D-3F, the main housing 110 forms an interior having a proximal port 120 for receiving a medical instrument (e.g., a Luer, needleless syringe, blunt cannula, etc.), a proximal cavity 115, and a distal port 130. The connector 100 has an open mode that permits fluid flow through the connector 100 (e.g., between the proximal port/inlet 120 and the distal port/outlet 130), and a closed mode that prevents fluid flow through the connector 100 (e.g., between the proximal port/inlet 120 and the distal port/outlet 130). To that end, the interior contains a valve mechanism 150 that selectively controls (i.e., allow/permits) fluid flow through the connector 100. The fluid passes through a complete fluid path 160 that extends between the proximal port 120, proximal cavity 115, and the distal port 130. To help secure and/or stabilize the device 100 on the patient during use, the device 100 may have a stabilization pad 117 extending from the housing 110. The stabilization pad 117 may have a curved surface that contours to the patient's skin.

It should be noted that although much of the discussion herein refers to the proximal port 120 as an inlet, and the distal port 130 as an outlet, the proximal and distal ports 120 and 130 also may be respectively used as outlet and inlet ports. Discussion of these ports in either configuration therefore is for illustrative purposes only. Alternatively, the main housing 110 may have a closed proximal end, instead of a proximal port, that surrounds a portion of the proximal cavity 115 to form the proximal portion of fluid path 160.

As mentioned above, within the interior of the housing 110 (e.g., within the fluid path 160), the connector 100 has an elastomeric valve member 150 that seals the proximal port 120. The valve member 150 may include a proximal portion 152 (e.g., a septum) and a valve wall 156 that extends distally from the proximal portion 152 within the interior of the housing 110 (e.g., within the proximal cavity 115). As shown in FIGS. 3D and 3F, the valve wall 156 forms a valve interior 158 within the interior of the housing 110. The valve member 150 also has a distal end 157 that preferably is open to form a distal port 159 into the valve interior 158. To help support the valve member 150 within the interior of the housing 110, the housing 110 (e.g., an outlet housing 112), may have shelf on which the distal end 157 of the valve member 150 may sit.

In some embodiments, the proximal portion 152 (e.g., the septum) of the valve member 150 may be flush with or extend slightly above an exterior proximal opening face 125 of the proximal opening 120 of the housing 110. The proximal portion 152 of the valve member 150 and the exterior inlet face 125 thus present a swabbable surface, i.e., it may be easily wiped clean with an alcohol swab, for example, or other swab.

As discussed above, a medical implement may be connected to the proximal opening 120 to allow a user to transfer fluid to and/or from a patient. To that end, the valve member 150 includes a resealable aperture 153 extending through the proximal portion 152. Among other things, the aperture 153 may be a pierced hole or a slit. Alternatively, the proximal portion 152 may be molded with the aperture 153. When the valve member 150 is in a closed mode (i.e., preventing passage of fluid), as shown in FIGS. 3A-3F, the aperture 153 may be held closed by the inner surface of the proximal opening 120. In that case, the inner diameter of the proximal opening 120 may be smaller than the outer diameter of the proximal portion 152 and thus, the housing 110 (e.g., the portion near the proximal opening 120) squeezes the aperture 153 closed. Alternatively, the valve member 150 (e.g., the proximal portion 152) may be formed so that the aperture 153 normally stays closed in the absence of radially inward force provided by the inner diameter of the proximal opening 120. In other words, the proximal portion 152 may be formed so that the aperture 153 normally is closed.

During operation (e.g., when transferring fluid to and/or from the patient via the proximal port 120), the medical practitioner may insert the medical implement into the proximal opening/port 120 of the housing 110. As the medical implement is inserted, the valve member 150, which normally closes the proximal opening 120, moves/deforms distally within the interior of the housing 110. As the valve member 150 continues to move/deform distally into the housing interior, the aperture 153 will open (e.g., when the proximal portion 152 enters the larger inner diameter portion of the inlet housing 114) to create fluid communication between the medical implement and the valve interior 158. Conversely, when the medical implement is withdrawn from the proximal opening 120 (e.g., after fluid transfer is complete), the elastomeric properties of the valve member 150 cause the valve member 150 to begin to move proximally within the interior of the housing and return to its at-rest position with the proximal portion 152 within (and closing) the proximal opening 114.

The outside surface of the proximal port 120 may also have inlet threads 90 for connecting the medical instrument. Alternatively or in addition, the proximal end may have a slip design for accepting instruments that do not have a threaded interconnect. In a similar manner, the distal end of the housing 110 has a skirt 170 containing threads 172 (see FIGS. 3D and 3F) for connecting a threaded port of another medical device (e.g., a catheter) or a medical instrument, to the distal port 130. The proximal end inlet threads 90 and the distal end threads 172 preferably comply with ANSI/ISO standards (e.g., they are able to receive/connect to medical instruments complying with ANSI/ISO standards). In addition to the threads described above, the internal geometry of the inlet housing 114 (may taper in an opposite direction to that of a standard luer taper.

As noted above, the conduit/connector 100 may have an entry channel 140 extending through the housing 100 and to which the tubing set 200 may be fluidly connected. As best shown in FIG. 3F, the entry channel 140 may extend through a wall of the outlet housing 112 and into the interior of the connector 100 below (e.g., distal to) the proximal cavity 115 and/or valve member 150. The first end 212 of the tube 210 be secured to and/or within the entry channel (e.g., via gluing, welding, press-fit, etc.) in order to fluidly connect the interior of the housing 110 and the tubing set 200. Therefore, as fluid flows through the tubing set 200 (e.g., from a medical implement and/or medical device connected to the connector 220), the fluid will enter the interior of the housing 110 below the valve member 150 and proximal cavity 115.

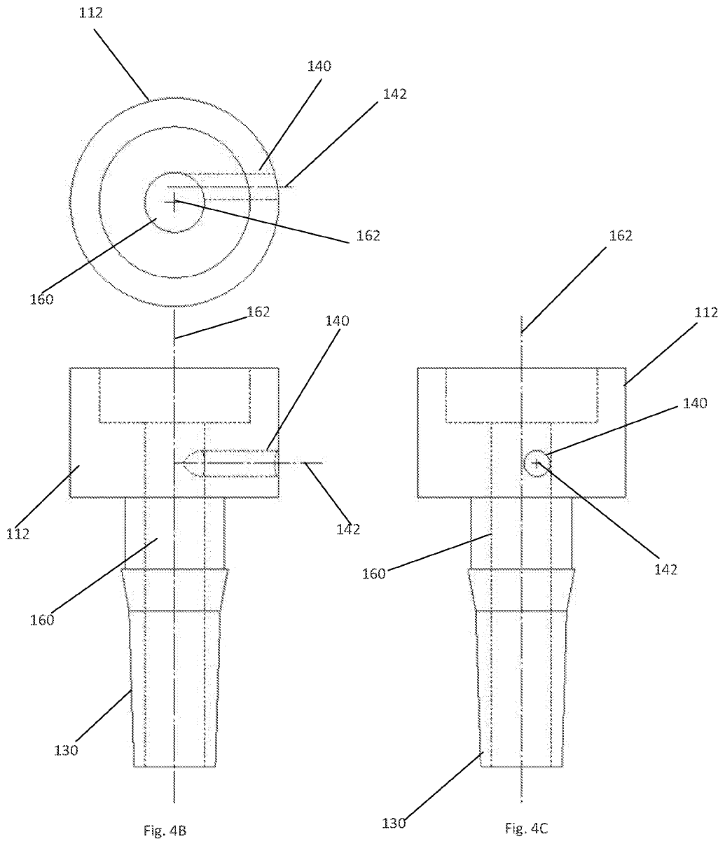

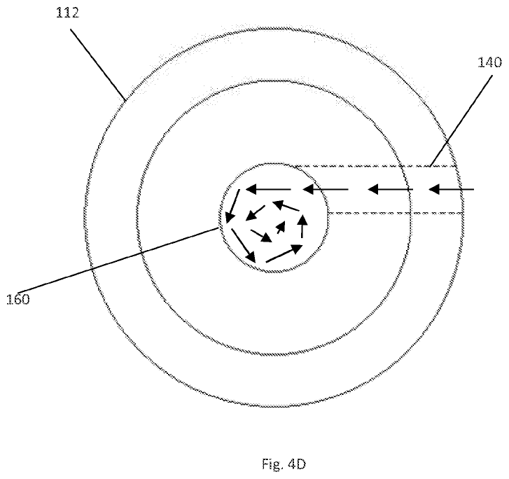

As noted above, various embodiments of the present invention are “self-flushing,” meaning that as fluid enters the housing 110 via the entry channel 140, it sufficiently flushes the interior of the housing 110 and valve interior 158 and/or proximal cavity 115 (e.g., the dead space within the prior art T-connector). To that end and as best shown in FIGS. 3E and 4A-4D, the entry channel 140 may be offset from the center of the housing 110 (e.g., the outlet housing 112) and the fluid path 160 of the housing 110. For example, the entry channel 140 may have a radial longitudinal axis 142 that is offset from the longitudinal axis 162 of the fluid path 160 of the housing 110 such that it does not intersect the longitudinal axis 162 of the fluid path 160 of the housing 110 (FIGS. 4B and 4C).

The offset between the entry channel 140 and the fluid path 160 causes the fluid entering the housing 110 via the tubing set 200 to enter on one side of the valve interior and begin a “swirl” type motion within the interior of the housing 110 (e.g., within the fluid path 160) (see FIG. 4D). At least a portion of the swirling fluid, in turn, interacts with the inner walls of the housing 110, causing at least a portion of the fluid to be directed proximally toward the proximal cavity 115 and, if equipped with a valve member 150, into and flushing the valve interior 158 (e.g., the fluid will be directed into the dead zone). To further help with flushing, in some embodiments, the angle between the entry channel 140 and the fluid path 160 may not be orthogonal. For example, the entry channel 140 may be angled upwards/proximally towards the valve interior 158 to direct the fluid into the valve interior 158 and/or proximal cavity 115.

It is important to note that the amount of the offset may vary depending on the application and the amount flushing required. For example, in some embodiments, the entry channel 140 (and the tube 210 of the tubing set 200) may offset from the fluid path 160 such that the outer diameter of the entry channel 140 is tangent (or nearly tangent) to the diameter of the fluid path 160. In such a configuration, the diameter of the entry channel 140 is preferably less than the diameter of the fluid path 140. In illustrative embodiments, this relationship may range from 1:2 to 1:4 (entry channel 140 entry diameter to fluid path 160 diameter). Alternatively, as shown in FIG. 4C), the offset may be such that the outer diameter of the entry channel 140 is located inward or outward of the outer diameter of the fluid channel 140.

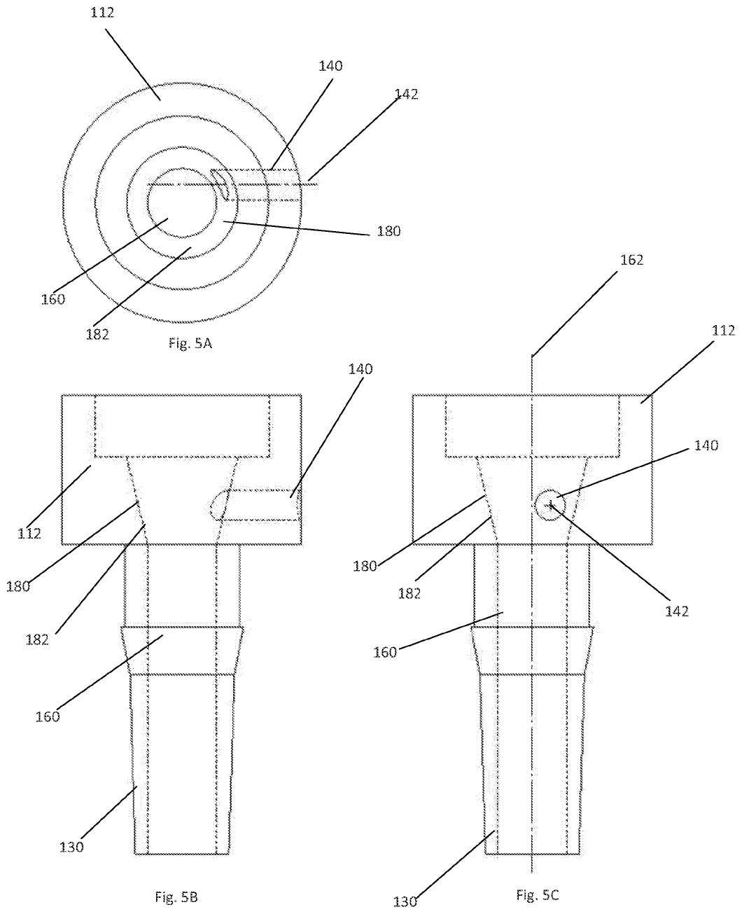

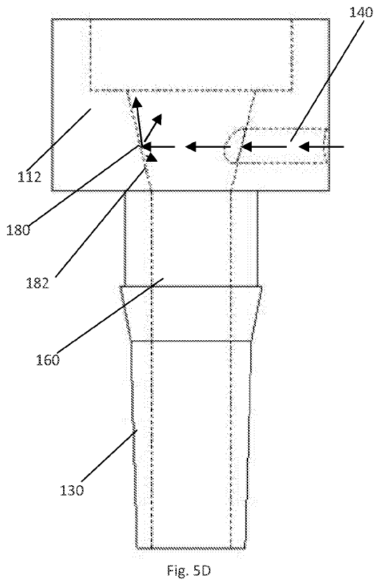

In addition to the offset entry channel 140, some embodiments may have additional features (e.g., flow directing features) that help direct the fluid entering the housing 110 via the entry channel 140 into the valve interior 158 and/or proximal cavity 115 to increase flushing. For example, the outlet housing 112 may include a conical or frusto-conical portion 180 portion with an inner diameter that decreases towards the distal end (e.g., towards the distal port/outlet 130) (FIGS. 5A-5D). As best shown in FIG. 5D, the surface 182 (e.g., the angled surface) of the conical portion 180 acts a contact surface against which fluid entering the housing 110 via the entry channel 140 impinges. As the fluid contacts/impinges on the contact surface 182, the surface 182 acts to redirect the fluid proximally toward the proximal cavity 115 and/or valve 150 and into the valve interior 158 and/or proximal cavity 115. This, in turn, increases the flushing within the device 100.

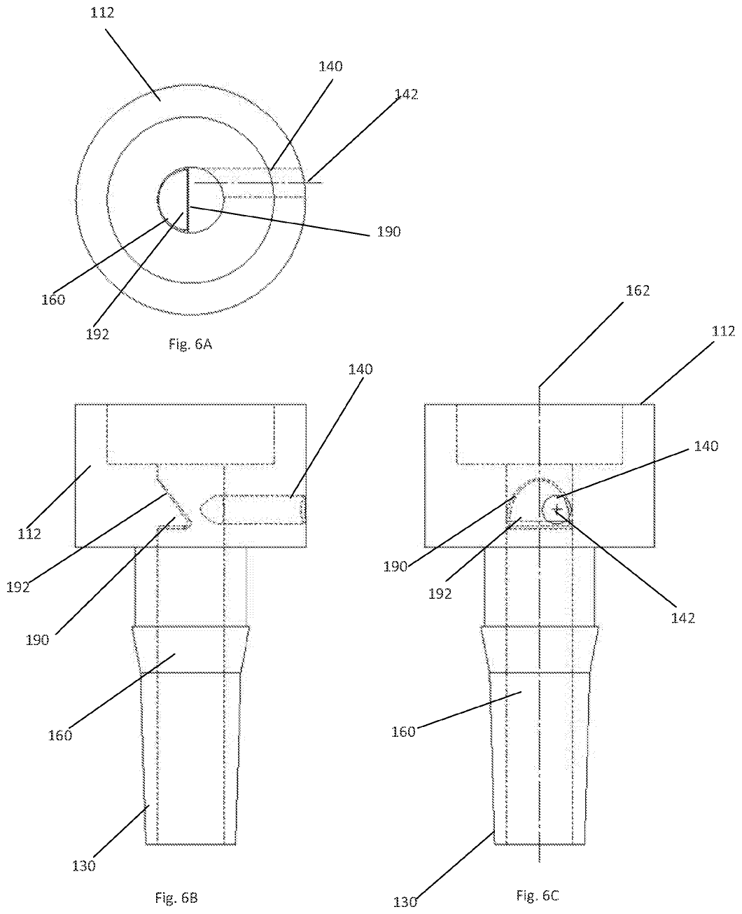

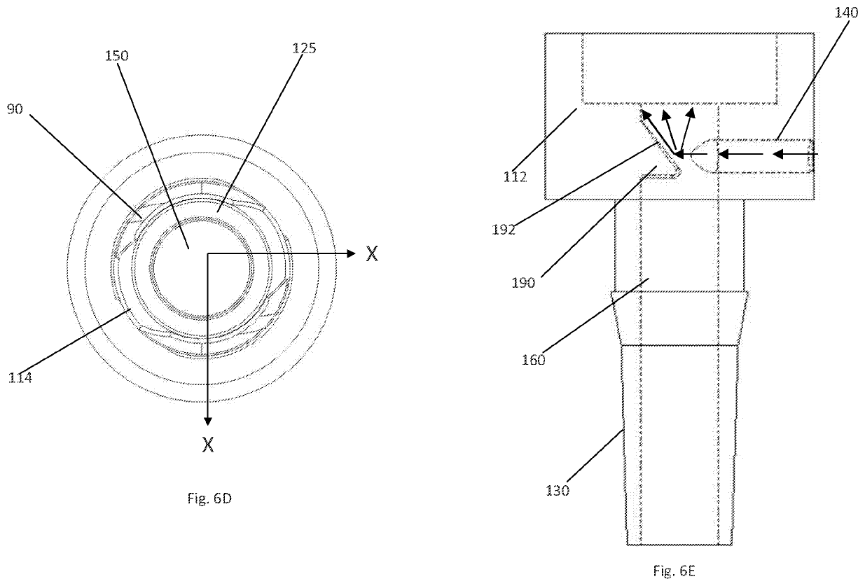

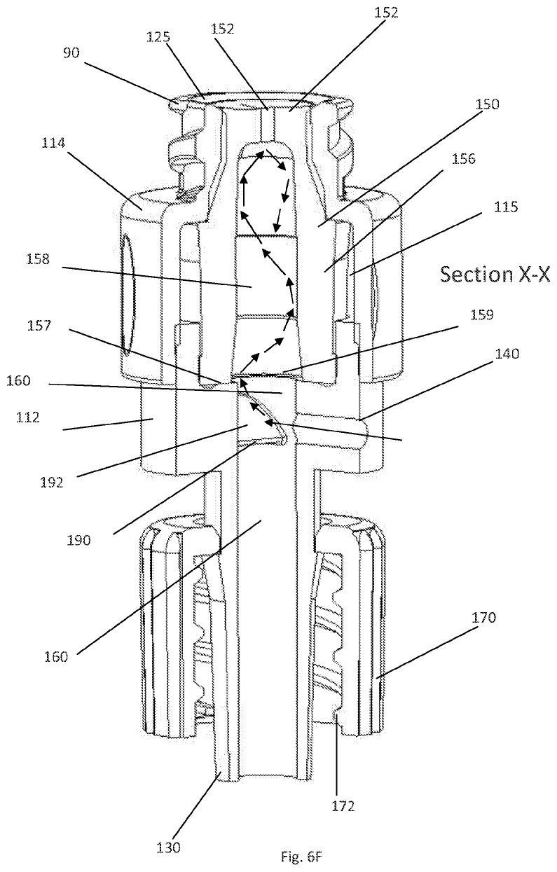

In addition to or instead of the conical portion 180, some embodiments may have a ramp 190 located within the flow path 160. The ramp 190 may extend around a portion (e.g., 180 degrees) of the inner diameter of the outlet housing 112 (e.g., in the fluid path 160) and extend proximally toward the proximal cavity 115 and valve interior 158 to create an angled surface 192 upon which the fluid entering the housing 110 impinges. To that end, in a manner similar to that described above for the conical portion 180, the fluid entering the housing 110 via the entry channel/tubing set fluid channel 140 will contact the angled surface 192 and flow proximally up the ramp 190 (see FIG. 6F). This, in turn, will redirect the fluid proximally toward the proximal cavity 115 and/or valve 150 and into the valve interior 158 and increase the flushing within the device 100.

It should be noted that, although FIGS. 6A-6F show the ramp 190 extending out from the inner wall of the fluid path 160 within the outlet housing 112 and around approximately 180 degrees of the diameter of the fluid path 160 (FIG. 6A), other embodiments can utilize different ramp configurations. For example, some embodiments may utilize ramps that extend more than 180 degrees around the fluid path 160 and other embodiments may utilize ramps that extend less than 180 degrees around the fluid path 160. Additionally or alternatively, instead of the ramp 190 extending into the fluid path 160, the ramp 180 may be recessed into the inner wall of the fluid path 160.

It should also be noted that other flow directing features may be used in addition to or instead of the conical portion 180 and ramp 190 discussed above. For example, some embodiments may include a shelf (not shown) that extends into the flow path 160 distal to the entry channel 140. The top surface of the shelf may act as the contact surface and essentially create a flow restriction that deters the fluid from flowing distally towards the outlet 130 and help to redirect the fluid proximally toward the proximal cavity 115 and/or valve interior 158.

In order to minimize flow disruption and restriction between the inlet 120 and outlet 130, it is preferable that each of the flow directing structures above protrude minimally from the fluid path diameter. This allows the main flow path 160 of the “T” to be open (straight through the main lumen), but also maintaining the ability to direct flow toward the valve interior 158 and/or proximal cavity 115. Alternatively, as noted above for the ramp 190, the flow directing feature may be formed by a recessed geometry within the wall of the main lumen.

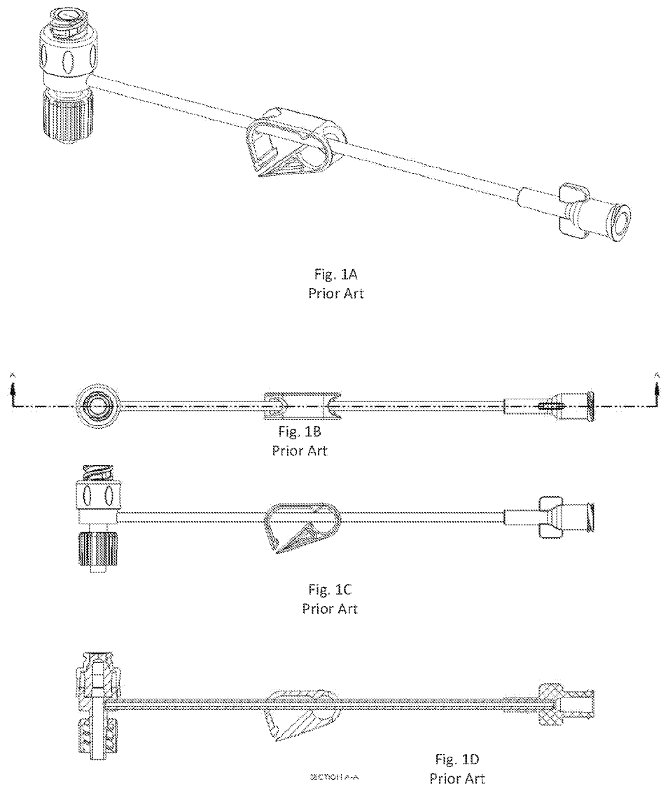

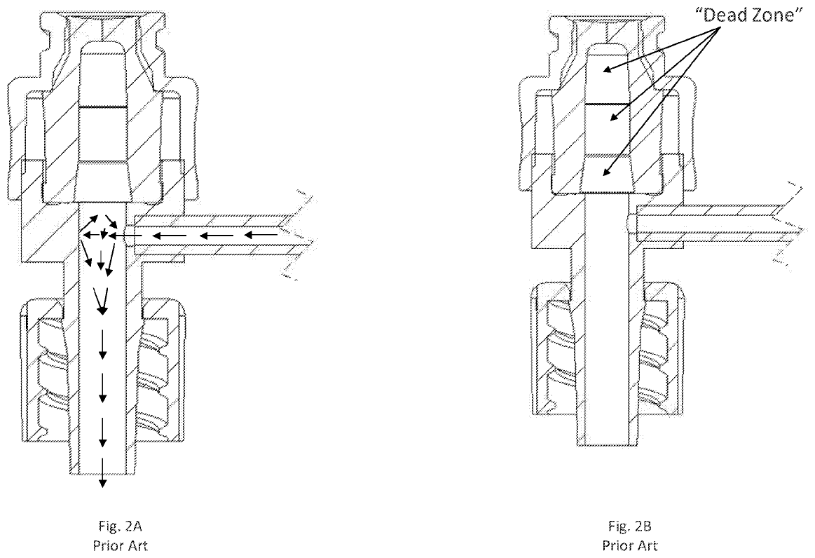

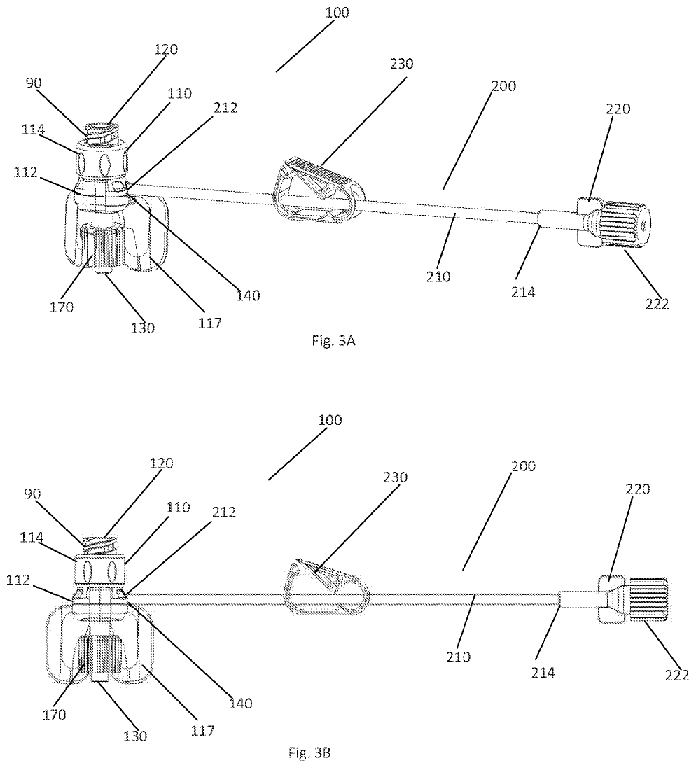

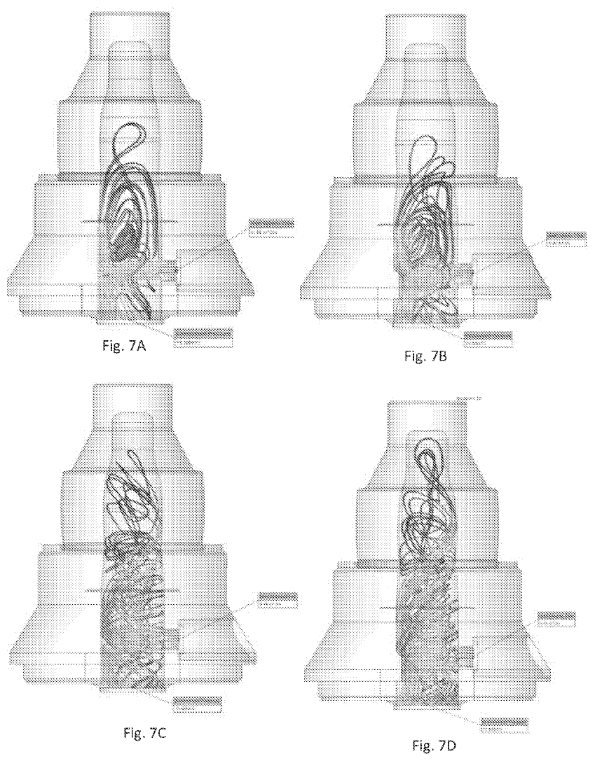

It should be understood that the various embodiments of the device 100 described above provide numerous advantages over prior art devices. Among others, under expected fluid flow rates, the design urges more fluid proximally to more fully flush the interior of the valve interior 158 and/or proximal cavity 115. For example, FIGS. 7A-7D show a series of fluid flow analyses to show the impact of the illustrative embodiments of the invention. FIG. 7A shows fluid flow through a centered extension/tubing set lumen into the main lumen/fluid path (e.g., a prior art system like that shown in FIGS. 1A-1D and 2A-2B). FIG. 7B shows fluid flow through a centered extension set lumen with the flow diverting feature (e.g., a conical portion, ramp, shelf, etc.) within the main lumen/flow path. FIG. 7C shows fluid flow through an offset extension/tubing set lumen into the main lumen/fluid path. FIG. 7D shows fluid flow through an offset extension/tubing set lumen into the main lumen/fluid path containing the flow diverting feature. As can be seen in these figures, fluid flow into the valve interior 158 and/or proximal cavity 115 and fluid flushing vastly improves with the addition of the features discussed above.

The embodiments of the invention described above are intended to be merely exemplary; numerous variations and modifications will be apparent to those skilled in the art.

Such variations and modifications are intended to be within the scope of the present invention.

1. A fluid conduit for use in a medical line, the fluid conduit comprising:
a housing having a body forming an interior, the housing further having a proximal cavity within the interior, an outlet, and a fluid path within the interior and extending between the cavity and outlet, the fluid path having a fluid path longitudinal axis;
an entry channel extending through the body of the housing distal to the cavity, the entry channel having a radial longitudinal axis and being offset from the fluid path such that the radial longitudinal axis of the entry channel does not intersect the longitudinal axis of the fluid path, thereby causing at least a portion of a fluid entering the interior of the housing via the entry channel to initiate a swirl-like motion within the interior of the housing; and
a contact surface within the fluid path, at least a portion of the contact surface located distal to the cavity and intersecting the radial longitudinal axis, the contact surface configured to direct at least a portion of the fluid entering the interior of the housing via the entry channel proximally into the cavity.

2. A fluid conduit according to claim 1, wherein the entry channel is configured at an angle such that fluid entering the interior of the housing is directed proximally towards the cavity.

3. A fluid conduit according to claim 1, further comprising a tubing set, the tubing set including:
a tube with a first end and a second end, the tube fluidly connected to the entry channel at the first end; and
a medical connector located at the second end and configured to connect to a medical implement.

4-5. (canceled)

6. A fluid conduit according to claim 1, wherein the contact surface is angled.

7. A fluid conduit according to claim 1, wherein the fluid path includes a conical portion having an inner diameter that decreases towards a distal end, the contact surface located on the conical portion.

8. A fluid conduit according to claim 1, further comprising:
a ramp located within the fluid path, the contact surface located on the ramp.

9-10. (canceled)

11. A fluid conduit according to claim 8, wherein the ramp is configured at an angle such that fluid entering the interior of the housing is directed proximally towards the cavity.

12. (canceled)

13. A fluid conduit according to claim 1, further comprising a shelf located within the fluid path and distal to the entry channel, the contact surface located on a top surface of the shelf.

14. A fluid conduit according to claim 1, further comprising:
a stabilization pad extending from at least a portion of the housing, the stabilization pad configured to stabilize the fluid conduit on a patient during use.

15. A fluid conduit according to claim 1, further comprising:
a valve member located within the housing interior and having a septum configured to close a proximal opening when the valve member is in a closed mode and a valve wall extending from the septum and forming a valve interior.

16. A fluid conduit according to claim 15, wherein the housing includes an inlet housing and an outlet housing, the valve member located at least partially within the inlet housing, the entry channel extending through a wall of the outlet housing.

17. A fluid conduit according to claim 15, wherein the offset and the contact surface cause the at least a portion of the fluid to flow into the valve interior.

18. A method of flushing a fluid conduit comprising:
providing a fluid conduit, the fluid conduit including
a housing having a body forming an interior, the housing further having a cavity within the interior, an outlet, and a fluid path within the interior, the fluid path extending between the cavity and outlet and having a fluid path longitudinal axis,
an entry channel extending through the body of the housing and distal to the cavity, the entry channel having a radial longitudinal axis and being offset from the fluid path such that the radial longitudinal axis of the entry channel does not intersect the longitudinal axis of the fluid path, the entry channel fluidly connected to a tubing set having a tube and a medical connector, and
a contact surface within the fluid path, at least a portion of the contact surface located distal to the cavity and intersecting the radial longitudinal axis;
connecting a medical implement to the medical connector; and
introducing a fluid into the fluid conduit via the tubing set and the entry channel, the offset causing at least a portion of the fluid entering the fluid conduit to initiate a swirl-like motion within the fluid path and contact the contact surface, thereby directing the at least a portion of the fluid proximally into the cavity.

19. A method according to claim 18, wherein the entry channel is configured at an angle such that fluid entering the interior of the housing via the entry channel is directed proximally towards the cavity.

20. A method according to claim 18, wherein the contact surface is angled.

21. A method according to claim 18, wherein the fluid path includes a conical portion, the contact surface located on the conical portion.

22. A method according to claim 18, wherein the fluid conduit further includes:
a ramp located within the fluid path, the contact surface located on the ramp, wherein the ramp is configured at an angle such that fluid entering the interior of the housing is directed proximally towards the cavity.

23-25. (canceled)

26. A method according to claim 18, wherein the fluid conduit further includes:
a valve member located within the housing interior and having a septum configured to close a proximal opening when the valve member is in a closed mode and a valve wall extending from the septum and forming a valve interior.

27. A method according to claim 26, wherein the housing includes an inlet housing and an outlet housing, the valve member located at least partially within the inlet housing, the entry channel extending through a wall of the outlet housing.

28. A method according to claim 26, wherein the offset and the contact surface cause the at least a portion of the fluid to flow into the valve interior.
